(19) United States
(12) Patent Application Publication
(10) Pub. No.: US 2022/0062461 A1
(43) Pub. Date: Mar. 3, 2022

(54) PHOTOSENSITIZER COMBINATION

(71) Applicants: University of Washington, Seattle, WA (US); Seattle Children's Hospital (dba Seattle Children's Research Institute), Seattle, WA (US)

(72) Inventors: James Chen, Seattle, WA (US); Tanner Clark, Seattle, WA (US); Thomas Lendvay, Seattle, WA (US)

(21) Appl. No.: 17/463,245

(22) Filed: Aug. 31, 2021

Related U.S. Application Data
(60) Provisional application No. 63/073,761, filed on Sep. 2, 2020.

(51) Int. Cl. A61L 2/08; A61L 2/26; C09B 67/22; G01N 21/31

(52) U.S. Cl. CPC A61L 2/088; A61L 2/26; A61L 2202/11; G01N 21/314; C09B 67/0033

(57) ABSTRACT

Methods for making disinfecting compositions based on phototherapy, systems for use in disinfecting with a combination of photosensitizers, and the disinfecting compositions themselves, are described. Concentrations of the photosensitizers can be based on the particular light source and the wavebands or fluence rates emitted by the light source for maximum singlet oxygen generation. Concentrations of the photosensitizers can also be based on the quantum yield of the photosensitizers.

### CROSS-REFERENCE(S) TO RELATED APPLICATION(S)

This application claims the benefit of U.S. Provisional Application No. 63/073,761, filed on Sep. 2, 2020, the disclosure of which is fully incorporated herein expressly for all purposes.

### BACKGROUND

Photosensitizers when activated by absorbed light produce singlet oxygen from molecular oxygen, as well as other reactive species, a process known as Photodynamic Therapy (PDT). Research has shown that photosensitizers such as methylene blue when activated by red light can generate singlet oxygen which is capable of irreversibly damaging viral particles by combining with, and essentially oxidizing viral components, rendering the virus particles non-infectious. However, there is a need for the development of new compositions that may generate greater amounts of singlet oxygen or that can achieve greater antimicrobial efficacies currently available using a single photosensitizer.

### SUMMARY

In an embodiment, a disinfection system comprises a light source that emits different wavebands of light at different fluence rates; and an article incorporating a composition inside or on a surface of the article being exposed to the light source, wherein the composition includes a combination of at least two photosensitizers, wherein each of the at least two photosensitizers absorbs light of a different waveband emitted from the light source, and the photosensitizer that absorbs the light waveband having the highest fluence rate has a highest concentration in the composition.

In an example, the photosensitizer that absorbs the light waveband having the lowest fluence rate has a lowest concentration in the composition.

In an example, the composition comprises one or more photosensitizers in addition to the photosensitizer that absorbs the light waveband having the highest fluence rate, and the one or more photosensitizers have a concentration equal to or less than the photosensitizer that absorbs the light waveband having the highest fluence rate.

In an example, each of the at least two photosensitizers is associated with a quantum yield, and the photosensitizer with the highest concentration in the composition is based on the fluence rates of the light wavebands absorbed by the photosensitizers and the quantum yields of the photosensitizers.

In an example, the composition comprises more than one photosensitizers that each absorb light of a different waveband, and the concentrations of the more than one photosensitizers from higher to lower is in the order of higher to lower fluence rates of the wavebands absorbed by the more than one photosensitizers.

In an example, the composition has three different photosensitizers.

In an example, the composition has four different photosensitizers.

In an example, at least two photosensitizers are selected from the group consisting of methylene blue derivatives, methylene blue, xanthene dyes, xanthene dye derivatives, chlorophyll derivatives, tetrapyrrole structures, porphyrins, chlorins, bacteriochlorins, phthalocyanines, texaphyrins, prodrugs, aminolevulinic acids, phenothiaziniums, squaraine, boron compounds, transition metal complexes, hypericin, riboflavin, curcumin, titanium dioxide, psoralens, tetracyclines, flavins, riboflavin, riboflavin derivatives, erythrosine, erythrosine derivatives, indocyanine green, and rose bengal.

In an example, a concentration of each photosensitizer in the composition is from 0.01 μM to 1,000 μM.

In an example, the light source includes an artificial light source or sunlight.

In one embodiment, a composition comprises at least two photosensitizers selected from the group consisting of methylene blue, riboflavin, erythrosine, rose bengal, and indocyanine green.

In an example, the composition is a solution including water, saline, or an alcohol.

In an example, a concentration of each photosensitizer in the composition is from 0.01 μM to 1,000 μM.

In an example, a concentration of each photosensitizer in the composition is from 0.1 μM to 1,000 μM.

In an example, a concentration of each photosensitizer in the composition is from 1 μM to 1,000 μM.

In an example, a concentration of each photosensitizer in the composition is from 10 μM to 1,000 μM.

In an example, a concentration of each photosensitizer in the composition is from 100 μM to 1,000 μM.

In an example, the composition comprises at least three photosensitizers selected from the group consisting of methylene blue, riboflavin, erythrosine, rose bengal, and indocyanine green.

In one embodiment, a method for making a composition including two or more photosensitizers comprises obtaining a baseline antimicrobial efficacy of a baseline composition including a single photosensitizer at a given concentration and given light parameters including illumination time, fluence rate, and lux; making a combination composition including the single photosensitizer and one or more photosensitizers; testing the combination composition for antimicrobial efficacy under one of the conditions: a total concentration of photosensitizers of the combination composition is less than the given concentration of the baseline composition; an illumination time is less than the illumination time of the baseline composition; a fluence rate is less than the fluence rate of the baseline composition; and a lux is less than the lux of the baseline composition.

In an example, the method further comprises, when the antimicrobial efficacy of the combination composition tests less than the baseline antimicrobial efficacy, replacing a photosensitizer other than the single photosensitizer with a different photosensitizer, and retesting the combination composition for antimicrobial efficacy under one of the following conditions: a total concentration of photosensitizers of the combination composition is less than the given concentration of the baseline composition; an illumination time is less than the illumination time of the baseline composition; a fluence rate is less than the fluence rate of the baseline composition; and a lux is less than the lux of the baseline composition.

In an example, the method further comprises, when the antimicrobial efficacy of the combination composition tests greater than the baseline antimicrobial efficacy, making the combination composition into a disinfecting composition.

In one embodiment, a disinfection system comprises a light source that emits different wavebands of light at different fluence rates; and an article incorporating a composition inside or on a surface of the article being exposed to the light source, wherein the composition includes a combination of at least two photosensitizers, wherein each of the at least two photosensitizers absorbs light of a different waveband emitted from the light source.

In an example, the light source is a white light source.

In an example, the light source is an LED emitting light in a blue waveband, yellow-green wavebands, and red waveband, wherein the red waveband has a lowest fluence rate compared to the blue waveband and the yellow-green wavebands.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a flow diagram of a method of making a composition having two or more photosensitizers;

FIG. 2 is a flow diagram of a method of making a composition having two or more photosensitizers;

FIG. 3 is a diagrammatical illustration of an example of a disinfecting system including a light source and a pair of glasses with a photosensitizer composition incorporated with the glasses;

FIG. 4 is a diagrammatical illustration of an example of a disinfecting system including a light source and a mask with a photosensitizer composition incorporated with the mask;

FIG. 5 is a diagrammatical illustration of an example of a disinfecting system including a light source and a glove with a photosensitizer composition incorporated with the glove; and

FIG. 6 is a diagrammatical illustration of an example of a disinfecting system including a light source and a cap with a photosensitizer composition incorporated with the cap.

### DETAILED DESCRIPTION

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the FIGURES, can be arranged, substituted, combined, separated, and designed in a wide variety of different configurations, all of which are explicitly contemplated herein.

Furthermore, the particular arrangements shown in the FIGURES should not be viewed as limiting. It should be understood other embodiments may include more or less of each element shown in a given FIGURE. Further, some of the illustrated elements may be combined or omitted. Yet further, an example embodiment may include elements not illustrated in the FIGURES. As used herein, with respect to measurements, “about” means+/−5%.

The current disclosure details compositions and methods based on photodynamic therapy that is an effective disinfection technique with demonstrated utility against microbes, such as viruses and other pathogens.

It shall be understood that the term “microbial,” “microbe,” and variations, as used herein, refers to an infectious microorganism, pathogen, or agent, including one or more of a virus, viroid, bacterium, archaea, protists, protozoan, prion, fungus, toxin, or the like.

Photodynamic therapy uses one or more photosensitizers activated by light of any waveband, including, for example, visible light, infrared, and ultraviolet.

A photosensitizer is a compound that can generate at least singlet oxygen in response to light provided at particular wavebands or wavelengths and for a particular duration. Singlet oxygen is known by the chemical formula, ¹O₂. Photosensitizer compositions herein and in the FIGURES include, but are not limited to, all types of methylene blue derivatives and methylene blue itself, xanthene dyes and derivatives, chlorophyll derivatives, tetrapyrrole structures, porphyrins, chlorins, bacteriochlorins, phthalocyanines, texaphyrins, prodrugs such as aminolevulinic acids, phenothiaziniums, squaraine, boron compounds, various transition metal complexes, hypericin, riboflavin, curcumin, titanium dioxide, psoralens, tetracyclines, flavins such as riboflavin, riboflavin derivatives, erythrosine, erythrosine derivatives, rose bengal, indocyanine green, and the like.

In some examples, photosensitizer compositions are a combination of ones that are generally recognized as safe, and that are capable of absorbing light over a wide spectral range. Compositions including photosensitizers may be provided in solutions, gels, and powder (e.g. dry). The compositions may include one or more excipients, solvents, diluents, gelling agents, and the like, in addition to one or more photosensitizer. Solvent or diluents may include water, saline, alcohols, and the like.

Concentration of a photosensitizer in a composition whether as a single photosensitizer or in a combination of photosensitizers can range from 0.01 μM to 1,000 μM (“μM” is used to mean 1×10⁻⁶ moles per liter). Unless otherwise stated, concentrations have the units of micromoles per liter).

Photosensitizers when activated by absorbed light produce singlet oxygen from molecular oxygen, as well as other reactive species, a process known as Photodynamic Therapy (PDT). Singlet oxygen is capable of irreversibly damaging microbes, such a bacteria, viruses, and other pathogens by combining with, and essentially oxidizing microbial components, rendering the microbe particles non-infectious or inactive.

Light includes any ambient indoor or outdoor light including sunlight. Any type of light source including sunlight, ambient light, and/or artificial light, can be used that emits the proper wavebands or wavelengths of light that are effectively absorbed by the photosensitizers to cause singlet oxygen generation. The illumination time and intensity of light needed for adequate generation of singlet oxygen may be determined empirically, experimentally, and/or derived from known data. Light source examples herein and in the FIGURES can be comprised of light emitting diodes (LED), xenon lamps, fluorescent bulbs and tubes, incandescent light bulbs, electroluminescent devices, lasers, and the like, even including sunlight. Other known or contemplated light sources are not excluded in any fashion, and include all known wavelengths and wavebands known to lead to a photodynamic effect that generates singlet oxygen which is particular to the photosensitizer or combinations of different types and amounts of photosensitizers.

In one example, an effective amount of light corresponds to an exposure time that can range from 1 second to 2 hours, and the lux (lumen per square meter) can range from 10 to 50,000. In one example, a preferred exposure time is from 1 minute to 1 hour and a lux range from 100 to 10,000. In one example, the most preferred exposure time is from 5 minutes to 30 minutes, and a lux range from 100 to 10,000. In one example, the fluence rate of light or of any waveband can range from 1-200 mW/cm².

Examples of compositions containing two or more photosensitizers to achieve an advantage that a single photosensitizer alone does not possess are described. Examples of methods of making the compositions of two or more photosensitizers are also described.

The photosensitizer compositions can be used for the disinfection of articles, such as, but not limited to personal protective equipment, clothing, headwear, equipment, machinery, surfaces, and other inanimate objects.

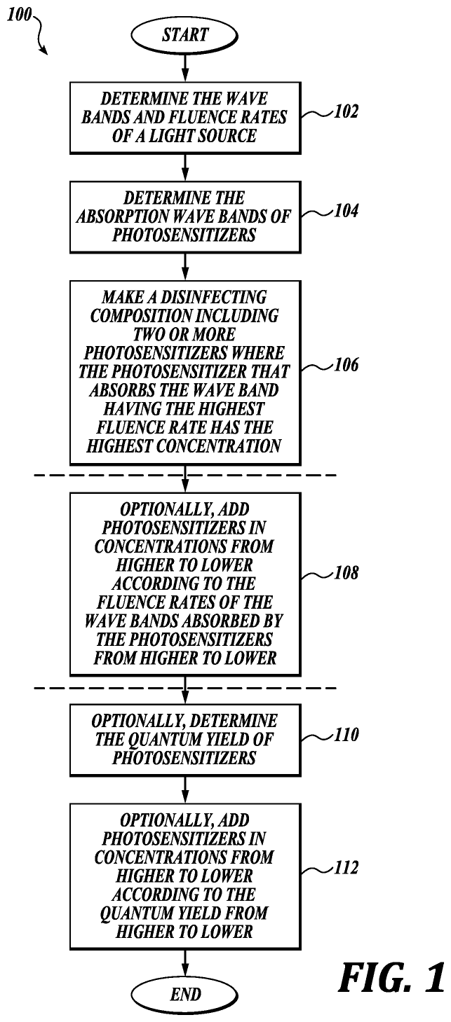

Referring to FIG. 1, one example of a method of making a disinfecting composition of two or more photosensitizers is diagrammed. The method includes determining the wavebands and fluence rates of the wavebands of a light source in block 102. Fluence rates of different wavebands and light sources can be known or determined by measuring.

From block 102, the method enters block 104. In block 104, the absorption wavebands of photosensitizers are determined. Some different photosensitizers have the property of absorbing light in different wavebands, particular to the molecular structure of each photosensitizer molecule. The absorption wavebands are known through the literature or experimentally. For example, it is known that methylene blue absorbs in the red waveband, riboflavin absorbs in the blue waveband, erythrosine absorbs in the green waveband, rose bengal absorbs in the yellow to green waveband, and indocyanine green absorbs in the infrared waveband. Photosensitizers can have different peak absorption wavelengths. Methylene blue has a peak absorption around 664 nanometers, erythrosine has a peak absorption around 530 nanometers, and riboflavin has a peak absorption wavelength of about 440 nm. Indocyanine green has an absorption waveband around 800 nm. From block 104, the method enters block 106.

In block 106, a disinfecting composition is made including two or more photosensitizers where the photosensitizer that absorbs the waveband having the highest fluence rate has the highest concentration in the composition. Additionally, the photosensitizer that absorbs the light waveband having the lowest fluence rate has the lowest concentration in the composition. In some examples, there may be two or more wavebands that have equal highest or lowest fluence rates, therefore, two photosensitizers that absorb the wavebands having the highest (or lowest) fluence rates can be provided in equal concentrations. In block 106, other photosensitizers can be added at the same or lower concentrations. In some examples, the concentrations of additional photosensitizers, other than the photosensitizer that absorbs the waveband having the highest fluence rate, can be dependent on the fluence rates of wavebands absorbed by the additional photosensitizers. For example, since white light contains wavebands encompassing the entire visible spectrum, a combination of photosensitizers that absorb the different wavebands of the white light can lead to a composition that is optimized at inactivating pathogenic virus particles in white light, providing for a composition having a broad spectrum of activity.

In an example, a white light can emit light in the red waveband at the highest fluence rate, followed by light in the green waveband, followed by light in the blue waveband. A composition based on the highest to lowest fluence rates of red, green, and blue wavebands can include methylene blue in the highest concentration, followed by erythrosine in the next highest concentration, followed by riboflavin in the next highest concentration. A composition of two or more photosensitizers in concentrations proportional to the fluence rates of the different wavebands can lead to generating effective amounts of singlet oxygen over a prolonged period of time.

In an example, a composition including two or more photosensitizers, the photosensitizer that absorbs the light waveband having the highest fluence rate has the highest concentration. In an example, additional photosensitizers are added at a lower concentration. In an example, additional photosensitizers can have the same concentration or the concentrations of the additional photosensitizers from higher to lower corresponding to the fluence rates of the wavebands going from higher to lower.

In an example, there tends to be less red light output in LED constructs intended for white light indoor and outdoor products, compared to blue and yellow-green light. Since methylene blue absorbs in the red waveband, and since there tends to be less available red light in white light LEDs, the methylene blue concentration and total amount can be less or the lowest, compared to riboflavin which absorbs in the blue waveband, and erythrosine which absorbs in the green waveband. So, one example of a composition which takes into account the lower amount of available red light of LEDs would be a ratio in grams of methylene blue to erythrosine to riboflavin of 1:2:2 respectively. Depending on the light source and fluence rates of particular wavebands, concentrations of the methylene blue, riboflavin, and erythrosine are formulated. White light created by LED combinations and constructs can incorporate varying ratios of red, green, and blue light, and exhibit variable spectral output distributions and characteristics leading to different concentrations of methylene blue, riboflavin, erythrosine, rose bengal optimized to the particular light source. When a light source emits in the infrared waveband, indocyanine green can be added in proportion to the fluence rate of infrared light.

In an example, a light source that emits a red waveband at the lowest fluence rate, a green waveband at a greater fluence rate than the red waveband, and a blue waveband greater than the red and green wavebands can lead to a concentration ratio of 1:2:3 of methylene blue to erythrosine to riboflavin. In an example, the ratio of photosensitizer concentrations corresponds to the ratio of the fluence rates of the wavebands absorbed by the photosensitizers.

In an example, the photosensitizer concentrations going from highest to lowest concentrations are based on fluence rates of wavebands absorbed by the photosensitizers going from highest to lowest fluence rates.

In the examples, concentrations of photosensitizer compositions can depend on the particular light source, the wavebands emitted by the light source, and the fluence rates.

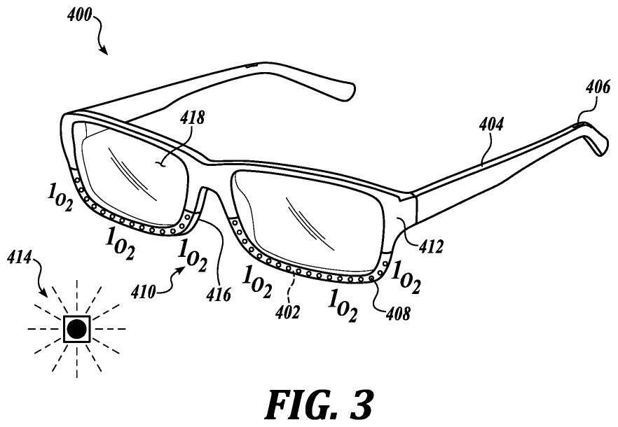

Accordingly, an example of a disinfection system herein and the FIGS. 3, 4, 5, and 6 can be configured to include a light source 414, 514, 614, and 714 that emits different wavebands of light at different fluence rates; and an article 400, 500, 600, and 700 incorporating a photosensitizer-containing composition 402, 502, 602, and 702 inside or on a surface of the article being exposed to the light source, wherein the composition includes a combination of at least two photosensitizers, wherein each of the at least two photosensitizers absorbs light of a different waveband emitted from the light source, and the photosensitizer that absorbs the light waveband having the higher fluence rate has the highest concentration in the composition.

An example of a disinfection system can be configured to include a light source 414, 514, 614, and 714 that emits different wavebands of light at different fluence rates; and an article 400, 500, 600, and 700 incorporating a composition 402, 502, 602, and 702 on the insider or on a surface of the article being exposed to the light source, wherein the composition includes a combination of at least two photosensitizers, wherein each of the at least two photosensitizers absorbs light of a different waveband emitted from the light source, and the photosensitizer concentrations from highest to lowest concentrations are based on fluence rates of wavebands absorbed by the photosensitizers going from highest to lowest fluence rates.

An example of a disinfection system can be configured to include a light source 414, 514, 614, and 714 that emits different wavebands of light at different fluence rates; and an article 400, 500, 600, and 700 incorporating a composition 402, 502, 602, and 702 on the inside or a surface of the article being exposed to the light source, wherein the composition includes a combination of at least two photosensitizers, wherein each of the at least two photosensitizers absorbs light of a different waveband emitted from the light source, and the ratio of photosensitizer concentrations corresponds to the ratio of the fluence rates of wavebands absorbed by the photosensitizers.

For any given light source, a composition of two or more photosensitizers can be designed where the photosensitizers are added dependent on or in proportion to the fluence rate of the waveband absorbed by the respective photosensitizers. In this manner, the total amount of photosensitizing drug in combination is minimized while maximizing singlet oxygen output by using more of the visible light spectrum. In other words, for a given amount of light, more photons will be utilized to generate singlet oxygen by using multiple photosensitizers, compared to using a single photosensitizer. In an example, prolonged singlet oxygen output is enabled by use of refillable containers and refillable application devices and tools which enable the photosensitizer combination to be reapplied as photobleaching, a process which essentially uses up the useful photosensitizer molecule, inevitably occurs.

From block 108, the method has the option to proceed to block 110. In block 110, the quantum yield of photosensitizers can be determined. The quantum yield is essentially the probability of singlet oxygen generation from the interaction of one absorbed photon with one photosensitizer molecule. The quantum yields are known from the experimental literature. For example, methylene blue is associated with a quantum yield of 0.52, riboflavin is associated with a quantum yield of 0.375 or higher depending on the test conditions, and erythrosine is associated with a quantum yield of around 0.6. In block 112, the concentrations of photosensitizers in the composition can also take into account the quantum yield of the photosensitizer. For example, the photosensitizers are added in concentrations from higher to low according to the highest to lowest quantum yield of the respective photosensitizers. In another example, quantum yield and the absorbed waveband can be considered in determining the photosensitizer concentration. For example, the photosensitizer concentrations are added in proportion to quantum yield and fluence rate.

In an example, it is possible to calculate singlet oxygen production from the molar concentrations of photosensitizers from which the number of photosensitizer molecules can be calculated. Then, the number of singlet oxygen molecules generated by different concentrations of photosensitizers in different combinations can be calculated, knowing the absorption spectrum of each photosensitizer. Various photosensitizer combinations are possible to be configured that are lower in concentration compared to a single photosensitizer, but the combination can generate an equivalent number of singlet oxygen molecules compared to a single photosensitizer. The photosensitizer combination can have a superior antiviral/antimicrobial effect, due to binding to different sites, prior to photoactivation, on the pathogen due to the different structures of the different photosensitizers. Also, there can be a propensity for self-shielding (which is essentially blocking of light) by the high concentration of a single photosensitizer which will reduce the efficiency of singlet oxygen generation by a single agent, which is absorbing light in a limited waveband, compared to a combination.

In an example, a composition including two or more photosensitizers, the photosensitizer that has the highest quantum yield has the highest concentration. In an example, additional photosensitizers are added at a lower concentration. In an example, additional photosensitizers can each have the same concentration or the concentrations of the additional photosensitizers from higher to lower correspond to the quantum yields going from higher to lower of the additional photosensitizers.

Accordingly, an example of a disinfection system can be configured to include a light source 414, 514, 614, and 714 that emits different wavebands of light at different fluence rates; and an article 400, 500, 600, and 700 incorporating a composition inside or on a surface of the article being exposed to the light source, wherein the composition includes a combination of at least two photosensitizers, wherein each of the at least two photosensitizers absorbs light of a different waveband emitted from the light source, and the photosensitizer that has the highest quantum yield has the highest concentration in the composition.

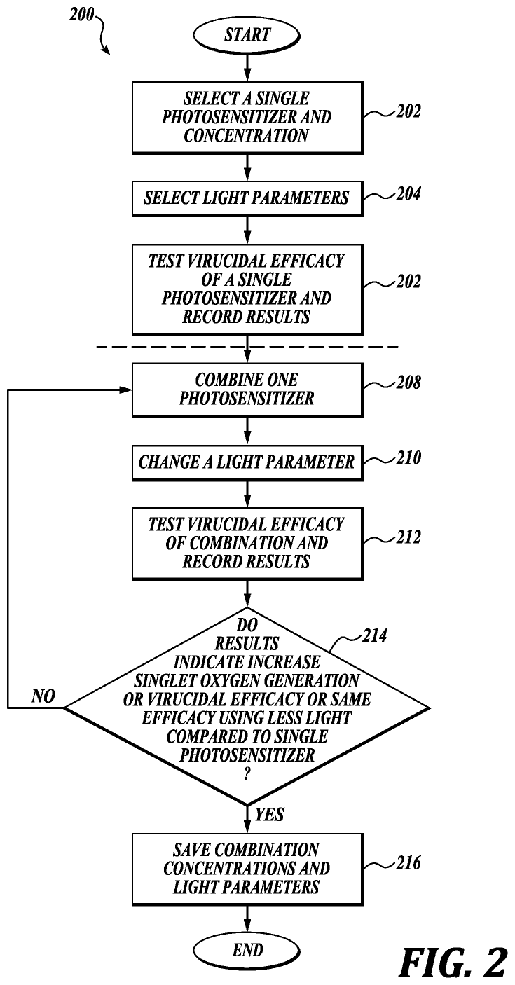

Referring to FIG. 2, one example of a method of making a disinfecting composition of two or more photosensitizers is diagrammed.

In block 202, a single photosensitizer and a concentration is selected for making a composition. In block 202, any of the photosensitizers can be used. A purpose of starting with a composition having a single photosensitizer it to obtain a baseline efficacy or a baseline of light parameters to compare with the efficacy of compositions of two or more photosensitizers. From block 202, the method proceeds to block 204.

In block 204, light parameters are selected. Light parameters include selecting the light source based on knowing the wavebands emitted from the light source and the fluence rates. A light parameter may also include the illumination time and the lux. From block 206, the method proceeds to block 206.

In block 206, testing for the antimicrobial efficacy of the baseline photosensitizer composition is conducted according to laboratory practices known in the art. A value representing the antimicrobial efficacy of the baseline composition is assigned according to practices known in the art. The value of antimicrobial efficacy forms a baseline of the composition having a single photosensitizer at a given concentration and at given light parameters, which are saved and referenced later as the baseline composition. The baseline antimicrobial efficacy of the single photosensitizer baseline composition can be compared to efficacies determined for the photosensitizer in combination with additional photosensitizers.

In an example, blocks 202, 204, and 206 are optional, for example, when there is pre-existing data or published data of the antimicrobial efficacy of a photosensitizer at a given concentration and at given light parameters. Blocks 202, 204, and 206 represent the baseline composition to which combinations of the baseline photosensitizer combined with additional photosensitizers will be compared.

Next, blocks 208, 210, and 212 can be used to develop a combination composition to compare with the baseline composition and baseline light parameters. In an example, combination compositions can determine whether lesser concentrations of photosensitizers in combination, with lower or higher total fluence rates, and with shorter or longer illumination time periods, may be superior to the teaching in the photodynamic art that higher photosensitizer concentrations, and higher total light fluence and longer illumination times are superior to the inverse. In testing, one variable can be changed at a time to determine what effect, if any, the variable has on the antimicrobial efficacy.

In block 208, photosensitizer of the baseline composition can be combined with one or more different photosensitizers. The combination of photosensitizers can have a combined concentration that is less than the concentration of the single photosensitizer used to establish the baseline antimicrobial efficacy. From block 208, the method proceeds to block 210.

In block 210, one or more of the light parameters can optionally be changed. Various light conditions, including broadband white light, and/or wavebands of visible and near infrared light which match the absorption wavebands of the various photosensitizers can be changed. In an example, the light parameters for the combination can use a shorter illumination period or a lower fluence rate or a lower lux as compared to the illumination period, the fluence rate, and the lux of the baseline composition. In blocks 208 and 210, it may be preferred to change one variable at a time to determine the effect of the variable. From block 210, the method proceeds to block 212.

In block 212, the combination composition with at least one variable of concentration or light parameter that is different to the baseline composition is tested for antimicrobial efficacy in the same manner as the baseline composition to assign a value representing the antimicrobial efficacy of the combination composition. From block 212, the method proceeds to block 214.

Block 214 is generally to determine whether the combination composition using multiple photosensitizers has an advantage over the baseline composition using a single photosensitizer. In block 214, an advantage can be an increase in singlet oxygen molecule generation or improved antimicrobial efficacy of the combination case compared to the baseline case when concentration and light parameters are equal. In one example of an advantage, a combination composition can have a similar antimicrobial efficacy as compared to the baseline composition; however, the antimicrobial efficacy of the combination compositions uses less total photosensitizer concentration, lower illumination time, lower lux, or lower fluence rate, as compared to the baseline composition. In block 214, when the results do not indicate an advantage, blocks 208, 210, and 212 are continually being repeated to test new photosensitizer combinations of doublets, triplets, quadruplets, and quintuplets, etc. changing concentrations or light parameters for each different iteration of blocks 208, 210, and 212. In block 214, when the results indicate an advantage of a combination composition, the concentrations and light parameters are saved. The combination composition having an advantage over a single photosensitizer composition can be used in a disinfecting composition applied to articles. The combination composition can be combined with the particular light source that emits the light parameters that were determined during testing to provide an advantage.

In examples, photosensitizer compositions herein and the photosensitizer compositions 402, 502, 602, and 702 in the FIGS. 3, 4, 5, and 6 include combinations of two or more of the following: methylene blue (3,7-bis(Dimethylamino)-phenothiazin-5-ium chloride), riboflavin (7,8-Dimethyl-10-[(2S,3S,4R)-2,3,4,5-tetrahydroxypentyl]benzo[g]pteridine-2,4-dione), erythrosine (2-(6-Hydroxy-2,4,5,7-tetraiodo-3-oxo-xanthen-9-yl)benzoic acid), rose bengal (4,5,6,7-Tetrachloro-3′,6′-dihydroxy-2′,4′,5′,7′-tetraiodo-3H-spiro[2]benzofuran-1,9′-xanthen]-3-one), and indocyanine green (sodium 4-[2-[(1E,3E,5E,7Z)-7-[1,1-dimethyl-3-(4-sulfonatobutyl)benzo[e]indol-2-ylidene]hepta-1,3,5-trienyl]-1,1-dimethylbenzo[e]indol-3-ium-3-yl]butane-1-sulfonate).

A first combination herein includes methylene blue and riboflavin. The first combination can be a solution including water, saline, or an alcohol. The first combination can have each photosensitizer in a molar concentration from 0.01 μM to 1,000 μM. The first combination can have each photosensitizer in a molar concentration from 0.1 μM to 1,000 μM. The first combination can have each photosensitizer in a molar concentration from 1.0 μM to 1,000 μM. The first combination can have each photosensitizer in a molar concentration from 10 μM to 1,000 μM. The first combination can have each photosensitizer in a molar concentration from 100 μM to 1,000 μM.

A second combination herein includes methylene blue and erythrosine. The second combination can be a solution including water, saline, or an alcohol. The second combination can have each photosensitizer in a molar concentration from 0.01 μM to 1,000 μM. The second combination can have each photosensitizer in a molar concentration from 0.1 μM to 1,000 μM. The second combination can have each photosensitizer in a molar concentration from 1.0 μM to 1,000 μM. The second combination can have each photosensitizer in a molar concentration from 10 μM to 1,000 μM. The second combination can have each photosensitizer in a molar concentration from 100 μM to 1,000 μM.

A third combination herein includes methylene blue and rose bengal. The third combination can be a solution including water, saline, or an alcohol. The third combination can have each photosensitizer in a molar concentration from 0.01 μM to 1,000 μM. The third combination can have each photosensitizer in a molar concentration from 0.1 μM to 1,000 μM. The third combination can have each photosensitizer in a molar concentration from 1.0 μM to 1,000 μM. The third combination can have each photosensitizer in a molar concentration from 10 μM to 1,000 μM. The third combination can have each photosensitizer in a molar concentration from 100 μM to 1,000 μM.

A fourth combination herein includes methylene blue and indocyanine green. The fourth combination can be a solution including water, saline, or an alcohol. The fourth combination can have each photosensitizer in a molar concentration from 0.01 μM to 1,000 μM. The fourth combination can have each photosensitizer in a molar concentration from 0.1 μM to 1,000 μM. The fourth combination can have each photosensitizer in a molar concentration from 1.0 μM to 1,000 μM. The fourth combination can have each photosensitizer in a molar concentration from 10 μM to 1,000 μM. The fourth combination can have each photosensitizer in a molar concentration from 100 μM to 1,000 μM.

A fifth combination herein includes riboflavin and erythrosine. The fifth combination can be a solution including water, saline, or an alcohol. The fifth combination can have each photosensitizer in a molar concentration from 0.01 μM to 1,000 μM. The fifth combination can have each photosensitizer in a molar concentration from 0.1 μM to 1,000 μM. The fifth combination can have each photosensitizer in a molar concentration from 1.0 μM to 1,000 μM. The fifth combination can have each photosensitizer in a molar concentration from 10 μM to 1,000 μM. The fifth combination can have each photosensitizer in a molar concentration from 100 μM to 1,000 μM.

A sixth combination herein includes riboflavin and rose bengal. The sixth combination can be a solution including water, saline, or an alcohol. The sixth combination can have each photosensitizer in a molar concentration from 0.01 μM to 1,000 μM. The sixth combination can have each photosensitizer in a molar concentration from 0.1 μM to 1,000 μM. The sixth combination can have each photosensitizer in a molar concentration from 1.0 μM to 1,000 μM. The sixth combination can have each photosensitizer in a molar concentration from 10 μM to 1,000 μM. The sixth combination can have each photosensitizer in a molar concentration from 100 μM to 1,000 μM.

A seventh combination herein includes riboflavin and indocyanine green. The seventh combination can be a solution including water, saline, or an alcohol. The seventh combination can have each photosensitizer in a molar concentration from 0.01 μM to 1,000 μM. The seventh combination can have each photosensitizer in a molar concentration from 0.1 μM to 1,000 μM. The seventh combination can have each photosensitizer in a molar concentration from 1.0 μM to 1,000 μM. The seventh combination can have each photosensitizer in a molar concentration from 10 μM to 1,000 μM. The seventh combination can have each photosensitizer in a molar concentration from 100 μM to 1,000 μM.

An eighth combination herein includes erythrosine and rose bengal. The eighth combination can be a solution including water, saline, or an alcohol. The eighth combination can have each photosensitizer in a molar concentration from 0.01 μM to 1,000 μM. The eighth combination can have each photosensitizer in a molar concentration from 0.1 μM to 1,000 μM. The eighth combination can have each photosensitizer in a molar concentration from 1.0 μM to 1,000 μM. The eighth combination can have each photosensitizer in a molar concentration from 10 μM to 1,000 μM. The eighth combination can have each photosensitizer in a molar concentration from 100 μM to 1,000 μM.

A ninth combination herein includes erythrosine and indocyanine green. The ninth combination can be a solution including water, saline, or an alcohol. The ninth combination can have each photosensitizer in a molar concentration from 0.01 μM to 1,000 μM. The ninth combination can have each photosensitizer in a molar concentration from 0.1 μM to 1,000 μM. The ninth combination can have each photosensitizer in a molar concentration from 1.0 μM to 1,000 μM. The ninth combination can have each photosensitizer in a molar concentration from 10 μM to 1,000 μM. The ninth combination can have each photosensitizer in a molar concentration from 100 μM to 1,000 μM.

A tenth combination herein includes rose bengal and indocyanine green. The tenth combination can be a solution including water, saline, or an alcohol. The tenth combination can have each photosensitizer in a molar concentration from 0.01 μM to 1,000 μM. The tenth combination can have each photosensitizer in a molar concentration from 0.1 μM to 1,000 μM. The tenth combination can have each photosensitizer in a molar concentration from 1.0 μM to 1,000 μM. The tenth combination can have each photosensitizer in a molar concentration from 10 μM to 1,000 μM. The tenth combination can have each photosensitizer in a molar concentration from 100 μM to 1,000 μM.

An eleventh combination herein includes methylene blue, riboflavin, and erythrosine. The eleventh combination can be a solution including water, saline, or an alcohol. The eleventh combination can have each photosensitizer in a molar concentration from 0.01 μM to 1,000 μM. The eleventh combination can have each photosensitizer in a molar concentration from 0.1 μM to 1,000 μM. The eleventh combination can have each photosensitizer in a molar concentration from 1.0 μM to 1,000 μM. The eleventh combination can have each photosensitizer in a molar concentration from 10 μM to 1,000 μM. The eleventh combination can have each photosensitizer in a molar concentration from 100 μM to 1,000 μM.

A twelfth combination herein includes methylene blue, riboflavin, and rose bengal. The twelfth combination can be a solution including water, saline, or an alcohol. The twelfth combination can have each photosensitizer in a molar concentration from 0.01 μM to 1,000 μM. The twelfth combination can have each photosensitizer in a molar concentration from 0.1 μM to 1,000 μM. The twelfth combination can have each photosensitizer in a molar concentration from 1.0 μM to 1,000 μM. The twelfth combination can have each photosensitizer in a molar concentration from 10 μM to 1,000 μM. The twelfth combination can have each photosensitizer in a molar concentration from 100 μM to 1,000 μM.

A thirteenth combination herein includes methylene blue, riboflavin, and indocyanine green. The thirteenth combination can be a solution including water, saline, or an alcohol. The thirteenth combination can have each photosensitizer in a molar concentration from 0.01 μM to 1,000 μM. The thirteenth combination can have each photosensitizer in a molar concentration from 0.1 μM to 1,000 μM. The thirteenth combination can have each photosensitizer in a molar concentration from 1.0 μM to 1,000 μM. The thirteenth combination can have each photosensitizer in a molar concentration from 10 μM to 1,000 μM. The thirteenth combination can have each photosensitizer in a molar concentration from 100 μM to 1,000 μM.

A fourteenth combination herein includes methylene blue, erythrosine, and rose bengal. The fourteenth combination can be a solution including water, saline, or an alcohol. The fourteenth combination can have each photosensitizer in a molar concentration from 0.01 μM to 1,000 μM. The fourteenth combination can have each photosensitizer in a molar concentration from 0.1 μM to 1,000 μM. The fourteenth combination can have each photosensitizer in a molar concentration from 1.0 μM to 1,000 μM. The fourteenth combination can have each photosensitizer in a molar concentration from 10 μM to 1,000 μM. The fourteenth combination can have each photosensitizer in a molar concentration from 100 μM to 1,000 μM.

A fifteenth combination herein includes methylene blue, erythrosine, and indocyanine green. The fifteenth combination can be a solution including water, saline, or an alcohol. The fifteenth combination can have each photosensitizer in a molar concentration from 0.01 μM to 1,000 μM. The fifteenth combination can have each photosensitizer in a molar concentration from 0.1 μM to 1,000 μM. The fifteenth combination can have each photosensitizer in a molar concentration from 1.0 μM to 1,000 μM. The fifteenth combination can have each photosensitizer in a molar concentration from 10 μM to 1,000 μM. The fifteenth combination can have each photosensitizer in a molar concentration from 100 μM to 1,000 μM.

A sixteenth combination herein includes methylene blue, rose bengal, and indocyanine green. The sixteenth combination can be a solution including water, saline, or an alcohol. The sixteenth combination can have each photosensitizer in a molar concentration from 0.01 μM to 1,000 μM. The sixteenth combination can have each photosensitizer in a molar concentration from 0.1 μM to 1,000 μM. The sixteenth combination can have each photosensitizer in a molar concentration from 1.0 μM to 1,000 μM. The sixteenth combination can have each photosensitizer in a molar concentration from 10 μM to 1,000 μM. The sixteenth combination can have each photosensitizer in a molar concentration from 100 μM to 1,000 μM.

A seventeenth combination herein includes riboflavin, erythrosine, and rose bengal. The seventeenth combination can be a solution including water, saline, or an alcohol. The seventeenth combination can have each photosensitizer in a molar concentration from 0.01 μM to 1,000 μM. The seventeenth combination can have each photosensitizer in a molar concentration from 0.1 μM to 1,000 μM. The seventeenth combination can have each photosensitizer in a molar concentration from 1.0 μM to 1,000 μM. The seventeenth combination can have each photosensitizer in a molar concentration from 10 μM to 1,000 μM. The seventeenth combination can have each photosensitizer in a molar concentration from 100 μM to 1,000 μM.

An eighteenth combination herein includes riboflavin, erythrosine, and indocyanine green. The eighteenth combination can be a solution including water, saline, or an alcohol. The eighteenth combination can have each photosensitizer in a molar concentration from 0.01 μM to 1,000 μM. The eighteenth combination can have each photosensitizer in a molar concentration from 0.1 μM to 1,000 μM. The eighteenth combination can have each photosensitizer in a molar concentration from 1.0 μM to 1,000 μM. The eighteenth combination can have each photosensitizer in a molar concentration from 10 μM to 1,000 μM. The eighteenth combination can have each photosensitizer in a molar concentration from 100 μM to 1,000 μM.

A nineteenth combination herein includes riboflavin, rose bengal, and indocyanine green. The nineteenth combination can be a solution including water, saline, or an alcohol. The nineteenth combination can have each photosensitizer in a molar concentration from 0.01 μM to 1,000 μM. The nineteenth combination can have each photosensitizer in a molar concentration from 0.1 μM to 1,000 μM. The nineteenth combination can have each photosensitizer in a molar concentration from 1.0 μM to 1,000 μM. The nineteenth combination can have each photosensitizer in a molar concentration from 10 μM to 1,000 μM. The nineteenth combination can have each photosensitizer in a molar concentration from 100 μM to 1,000 μM.

A twentieth combination herein includes erythrosine, rose bengal, and indocyanine green. The twentieth combination can be a solution including water, saline, or an alcohol. The twentieth combination can have each photosensitizer in a molar concentration from 0.01 μM to 1,000 μM. The twentieth combination can have each photosensitizer in a molar concentration from 0.1 μM to 1,000 μM. The twentieth combination can have each photosensitizer in a molar concentration from 1.0 μM to 1,000 μM. The twentieth combination can have each photosensitizer in a molar concentration from 10 μM to 1,000 μM. The twentieth combination can have each photosensitizer in a molar concentration from 100 μM to 1,000 μM.

A twenty-first combination herein includes methylene blue, riboflavin, erythrosine, and rose bengal. The twenty-first combination can be a solution including water, saline, or an alcohol. The twenty-first combination can have each photosensitizer in a molar concentration from 0.01 μM to 1,000 μM. The twenty-first combination can have each photosensitizer in a molar concentration from 0.1 μM to 1,000 μM. The twenty-first combination can have each photosensitizer in a molar concentration from 1.0 μM to 1,000 μM. The twenty-first combination can have each photosensitizer in a molar concentration from 10 μM to 1,000 μM. The twenty-first combination can have each photosensitizer in a molar concentration from 100 μM to 1,000 μM.

A twenty-second combination herein includes methylene blue, riboflavin, erythrosine, and indocyanine green. The twenty-second combination can be a solution including water, saline, or an alcohol. The twenty-second combination can have each photosensitizer in a molar concentration from 0.01 μM to 1,000 μM. The twenty-second combination can have each photosensitizer in a molar concentration from 0.1 μM to 1,000 μM. The twenty-second combination can have each photosensitizer in a molar concentration from 1.0 μM to 1,000 μM. The twenty-second combination can have each photosensitizer in a molar concentration from 10 μM to 1,000 μM. The twenty-second combination can have each photosensitizer in a molar concentration from 100 μM to 1,000 μM.

A twenty-third combination herein includes methylene blue, erythrosine, rose bengal, and indocyanine green. The twenty-third combination can be a solution including water, saline, or an alcohol. The twenty-third combination can have each photosensitizer in a molar concentration from 0.01 μM to 1,000 μM. The twenty-third combination can have each photosensitizer in a molar concentration from 0.1 μM to 1,000 μM. The twenty-third combination can have each photosensitizer in a molar concentration from 1.0 μM to 1,000 μM. The twenty-third combination can have each photosensitizer in a molar concentration from 10 μM to 1,000 μM. The twenty-third combination can have each photosensitizer in a molar concentration from 100 μM to 1,000 μM.

A twenty-fourth combination herein includes methylene blue, riboflavin, rose bengal, and indocyanine green. The twenty-fourth combination can be a solution including water, saline, or an alcohol. The twenty-fourth combination can have each photosensitizer in a molar concentration from 0.01 μM to 1,000 μM. The twenty-fourth combination can have each photosensitizer in a molar concentration from 0.1 μM to 1,000 μM. The twenty-fourth combination can have each photosensitizer in a molar concentration from 1.0 μM to 1,000 μM. The twenty-fourth combination can have each photosensitizer in a molar concentration from 10 μM to 1,000 μM. The twenty-fourth combination can have each photosensitizer in a molar concentration from 100 μM to 1,000 μM.

A twenty-fifth combination herein includes riboflavin, erythrosine, rose bengal, and indocyanine green. The twenty-fifth combination can be a solution including water, saline, or an alcohol. The twenty-fifth combination can have each photosensitizer in a molar concentration from 0.01 μM to 1,000 μM. The twenty-fifth combination can have each photosensitizer in a molar concentration from 0.1 μM to 1,000 μM. The twenty-fifth combination can have each photosensitizer in a molar concentration from 1.0 μM to 1,000 μM. The twenty-fifth combination can have each photosensitizer in a molar concentration from 10 μM to 1,000 μM. The twenty-fifth combination can have each photosensitizer in a molar concentration from 100 μM to 1,000 μM.

A twenty-sixth combination herein includes methylene blue, riboflavin, erythrosine, rose bengal, and indocyanine green. The twenty-sixth combination can be a solution including water, saline, or an alcohol. The twenty-sixth combination can have each photosensitizer in a molar concentration from 0.01 μM to 1,000 μM. The twenty-sixth combination can have each photosensitizer in a molar concentration from 0.1 μM to 1,000 μM. The twenty-sixth combination can have each photosensitizer in a molar concentration from 1.0 μM to 1,000 μM. The twenty-sixth combination can have each photosensitizer in a molar concentration from 10 μM to 1,000 μM. The twenty-sixth combination can have each photosensitizer in a molar concentration from 100 μM to 1,000 μM.

In an example, any number of pleasing scents, plant/fruit extracts that can be found in a variety of aromatherapy oils such as lavender, eucalyptus, lemon, orange, or peppermint can be optionally added to the photosensitizer compositions.

In an example, riboflavin or other photosensitizers can be incorporated into a hyaluronic acid formulation, with the concentration and volume of riboflavin or other photosensitizer determined using laboratory testing to optimize the riboflavin concentration and volume that kills virus in ambient light while protecting the skin from singlet oxygen. High molecular weight hyaluronic acids are known not to penetrate the skin, and therefore can be used in combination with a photosensitizer such as riboflavin, or in combination with other photosensitizers, as a topical disinfectant when exposed to ambient light. The lack of penetration of hyaluronic acid ensures that the riboflavin or other incorporated photosensitizers contained in the hyaluronic acid formulation remain external to the skin, and especially external to the outer skin cell layer called the stratum corneum. The stratum corneum is comprised of a layer of dead skin cells, which are not affected by a low concentration of singlet oxygen, rendering this topical disinfectant modality ideal for frequent hand disinfection. This is particularly useful for persons, adults, and children with delicate skin, or who have pre-existing skin damage or skin irritation but require hand sanitation.

In the illustrated examples, the compositions including combinations of two or more photosensitizers can be incorporated into wearable articles, such as clothing, personal protective equipment, or may be applied to surfaces of furniture, equipment, machinery, and the like, to disinfect the item or to provide protection from microbial infection. The compositions when combined with a particular light source designed having the absorption wavebands of the photosensitizers can provide useful disinfecting systems for many applications.

Referring to FIG. 3, an example article includes a pair of glasses 400. In the example, the glasses 400 are manufactured so as to enable containment of photosensitizer composition 402 in solution within one or more hollow frame components. The various components of the pair of glasses 400 such as the temples 404 and frame 412 can be made as hollow tubes which can be filled with a photosensitizer-containing solution 402. In one example, the temples 404 and lens frame 412 can be made from polymers or plastics which are opaque to light for the majority of the frame and temples with the exception of the lower (inferior) bottom portion 416 of the frame 412 which is translucent. Photosensitizer molecules in the light transmissible, translucent bottom portion of the frame 412 can be activated by a light source 414 and generate singlet oxygen 410.

In an example, the lower bottom portion of the frame 412 contains pores 410 through which the singlet oxygen molecules 410 can be transmitted. Singlet oxygen molecules 410 can travel into the air around the lower face. The singlet oxygen 410 in the air around the lower face can act as a shield, killing airborne viruses or other microbes before inhalation. The depot within the frame enables a continuous capillary filling of the lower frame portion 416 where photosensitizer-containing solution 402 can evaporate at the site of the pores 408 at the air interface. The pores 408 are sized such that singlet oxygen 410 generated by the light source 414 readily escapes, while surface tension is high enough to prevent dripping of the solution out of the pores 408.

In an example, the pore size and photosensitizer concentration in solution is tested and optimized in a series of laboratory testing such that the photosensitizer is continuously or intermittently delivered to the lower, light transparent portion of the frames, where singlet oxygen is produced, and photosensitizer solution evaporates and is renewed by capillary action, drawing more photosensitizer solution into the lower portion of the frames.

In an example, the lenses 418 of the pair of glasses 400 can be hollow which can contain photosensitizer solution in communication with the light transmissible lower frame portion. The set of hollow lenses 418 can be removed and refilled with a photosensitizer-containing solution, or the lenses 418 can be supplied for a single-use, are pre-filled, and are disposable lenses 418. The lenses 418 are optionally sized to be removable from the frame 412. A conduit in the frame 412 connects with an opening in the hollow lenses 418. For example, the hollow lenses 418 can incorporate an opening on their inferior, bottom aspect, which enables photosensitizer 402 to elute into the transparent bottom portion 416 of the glasses frame 412.

In an example, the pair of glasses 400 can be prefilled with the photosensitizer-containing solution, or filled as needed by way of a small opening 406 in the frame 412 or temple 404 that can be reversibly sealed.

In an example, the pair of glasses 400 contains a photosensitizer-containing solution of methylene blue, riboflavin, and erythrosine in a 1:1.5:1.5 concentration ratio. The solution can be injected through the small opening 406 which can accommodate a needle attached to a syringe containing a 3.0 ml photosensitizer-containing solution.

In an example, the solution can fill up the glasses' frame 412, the temples 404, and the lenses 418 of the glasses 400. The solution is exposed to any ambient light 414 including natural and artificial light at the transparent portion 416 of the frame 412 and singlet oxygen 410 is generated which escapes through the tiny pores 408 which have been drilled into the lowest, bottom section 416 of the glasses' frame 412 on both sides. A cloud or barrier of singlet oxygen is emitted over the lower half of the wearer's face that can inactivate virus or microbes which come in contact with the singlet oxygen molecules 410 upon inhalation and exhalation.

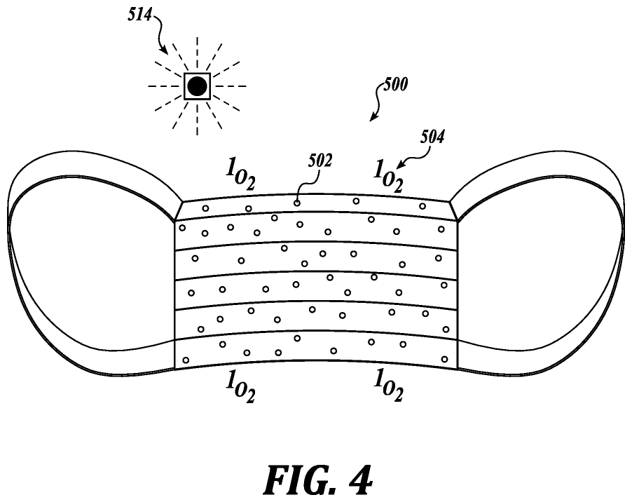

FIG. 4 is a diagrammatical illustration of a mask 500 used for personal protection. The mask 500 has a composition including a photosensitizer 502 incorporated on the surface and/or within the mask fabric. The photosensitizer 502 is activated by any suitable light source 514 of the proper waveband to generate singlet oxygen 504 in the areas close to the mask 500. Singlet oxygen 504 can form a cloud around the mask 500. Therefore, viruses and other microbes can be inactivated in the case where air may be inhaled by the wearer through the sides and top or bottom of the mask due to a loose or improper fit of the mask 500 to the skin surface. The singlet oxygen 504 may also inactivate any viruses or microbes exhaled by the wearer.

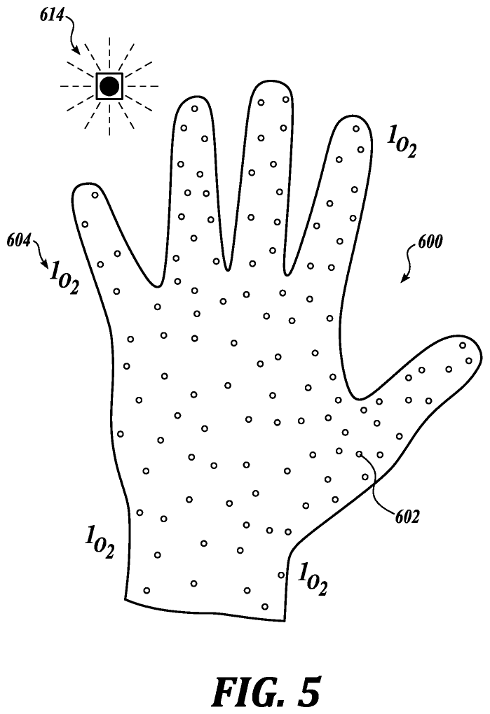

FIG. 5 is a diagrammatical illustration of a glove 600 for personal protection. The glove 600 has a composition including a photosensitizer 602 incorporated on the surface and/or within the glove fabric. The photosensitizer 602 is activated by any suitable light source 614 to generate singlet oxygen 604 in the vicinity to the glove 600 exterior. Therefore, viruses and other microbes can be inactivated in the case where the glove 600 is used to handle contaminated items or touches surfaces contaminated with viruses or other microbes.

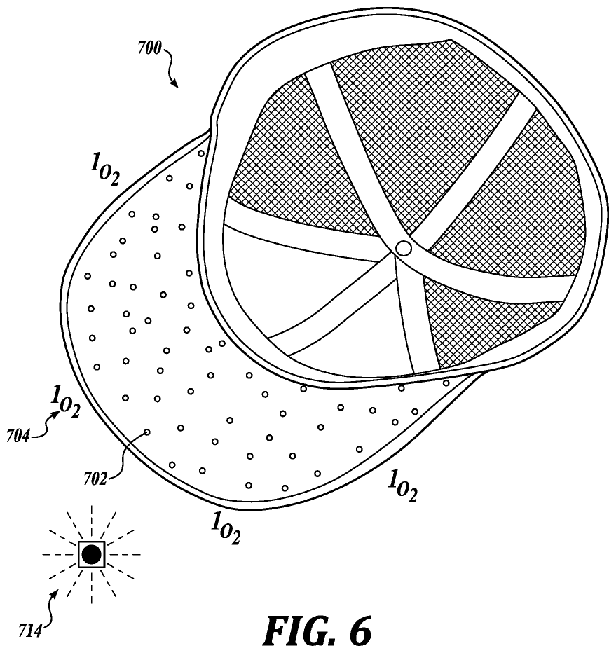

FIG. 6 is a diagrammatical illustration of a cap 700 for wearing. The cap 700 has a composition including a photosensitizer 702 incorporated on the surface and/or within the cap fabric. Particularly, the underside of the brim may be incorporated with the photosensitizer 702. The photosensitizer 702 is activated by any suitable light source 714 to generate singlet oxygen 704 in the area underneath the brim, and in proximity to the face, nose, and mouth of the wearer. Therefore, viruses and other microbes can be inactivated in the air being inhaled or exhaled by the cap wearer.

### Example 1

The hollow construction of a pair of glasses enables pre-filling of a photosensitizer solution of methylene blue, riboflavin, and erythrosine in a 1:1.5:1.5 concentration ratio. A total of 3.0 milliliters (ml) of a 10 micromolar solution of the photosensitizer solution using normal saline as the diluent is injected through a small opening which accommodates a needle attached to a syringe containing the 3.0 ml photosensitizer solution. The solution fills up the glasses' frame, the temples, and the lenses of the glasses. The solution is almost colorless, so vision by the wearer is not impaired. The solution is exposed to ambient light at the transparent bottom portion of the glasses frame, and singlet oxygen is generated which escapes through tiny pores which have been drilled into the lowest, bottom section of the glasses' frame on both sides. A cloud or barrier of singlet oxygen is emitted over the lower half of the wearer's face, inactivating virus which come in contact with the singlet oxygen molecules. Virus in the air proximate to the wearer's lower face is destroyed, which reduces the risk of pathogenic virus inhalation and infection. The photosensitizer solution slowly evaporates at the pore/air interface and undergoes photobleaching simultaneously. Capillary action draws more active photosensitizer solution to the light transparent lower portion of the glasses frame, so that singlet oxygen is continually generated and emitted into the air proximate to the wearer's lower face.

### Example 2

A series of laboratory tests can be conducted on the photosensitizer combination of methylene blue, riboflavin, and erythrosine in order to determine the optimal concentrations and volumes of each photosensitizer that emits a maximal amount of singlet oxygen molecules for a given ambient light intensity and fluence rate. The pore diameter and numbers that can be drilled into the lower portion of the glasses frame which is transparent to light is determined experimentally such that the pore diameter is the maximum size while retaining enough photosensitizer solution surface tension to prevent loss of photosensitizer solution by dripping.

### Example 3

A series of tests performed in a laboratory setting can be conducted in order to determine the inner configuration of the hollow tubes and inner hollow chambers of the glasses lenses that permits an optimal rate of flow due to capillary action of the photosensitizer solution towards the light-transmissible lower section of the pair of glasses frame.

### Example 4

The photosensitizer solution-containing lenses of a pair of glasses can be removable after being photobleached and depleted from photoactivation and evaporation and can be exchanged for a new pair of lenses containing a fresh photosensitizer solution. The replacement lenses are inserted in the frame in order to provide for further virucidal singlet oxygen production from the glasses.

### Example 5

The photosensitizer riboflavin can be tested at various concentrations and volumes with formulations of high molecular weight hyaluronic acid for an optimal balance of rapid antiviral activity in ambient light with optimal skin protection.

### Example 6

Combining various photosensitizers, such as methylene blue, riboflavin, rose bengal, erythrosine, indocyanine green, curcumin, bergamot, porphyrins, chlorins, texaphyrins, purpurins, psoralens, titanium dioxide, and other photosensitizers and photocatalysts can enhance the speed and quantity of singlet oxygen and other reactive species such as hydrogen peroxide, superoxide anion, hydroxyl radicals, and the like. A series of experiments can be performed, preferably combining combinations from methylene blue, riboflavin, rose bengal, erythrosine, and indocyanine green at doses ranging from 0.01, 0.1, 1.0, 10, 100, to 1000 micromolar concentrations, in doublets, triplets, and quadruplet combinations, and then comparing the combinations to single photosensitizer compositions. Various light conditions, including broadband white light, and/or wavebands of visible and near infrared light which match the absorption wavebands of the various photosensitizers, are utilized for illumination and photosensitization of viruses, including the SARS-CoV-2, and other pathogenic microbes including Staphylococcus aureus, in a series of experiments.

The experiments can be performed to determine whether lesser concentrations of photosensitizers in combination, with lower or higher total fluence rates, and with shorter or longer illumination time periods, may be superior to the teaching in the photodynamic art that higher photosensitizer concentrations, and higher total light fluence and longer illumination times are superior to the inverse.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments and examples of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments and examples can be combined or substituted for elements in other embodiments and examples. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

While illustrative embodiments have been illustrated and described, it will be appreciated that various changes can be made therein without departing from the spirit and scope of the invention.

The embodiments of the invention in which an exclusive property or privilege is claimed are defined as follows:

1. A disinfection system, comprising:
a light source that emits different wavebands of light at different fluence rates; and
an article incorporating a composition inside or on a surface of the article being exposed to the light source, wherein the composition includes a combination of at least two photosensitizers, wherein each of the at least two photosensitizers absorbs light of a different waveband emitted from the light source, and the photosensitizer that absorbs the light waveband having the highest fluence rate has a highest concentration in the composition.

2. The system of claim 1, wherein the photosensitizer that absorbs the light waveband having the lowest fluence rate has a lowest concentration in the composition.

3. The system of claim 1, wherein the composition comprises one or more photosensitizers in addition to the photosensitizer that absorbs the light waveband having the highest fluence rate, and the one or more photosensitizers have a concentration equal to or less than the photosensitizer that absorbs the light waveband having the highest fluence rate.

4. The system of claim 1, wherein each of the at least two photosensitizers is associated with a quantum yield, and the photosensitizer with the highest concentration in the composition is based on the fluence rates of the light wavebands absorbed by the photosensitizers and the quantum yields of the photosensitizers.

5. The system of claim 1, wherein the composition comprises more than one photosensitizers that each absorb light of a different waveband, and the concentrations of the more than one photosensitizers from higher to lower is in the order of higher to lower fluence rates of the wavebands absorbed by the more than one photosensitizers.

6. The system of claim 5, having three different photosensitizers.

7. The system of claim 5, having four different photosensitizers.

8. The system of claim 1, wherein the at least two photosensitizers are selected from the group consisting of methylene blue derivatives, methylene blue, xanthene dyes, xanthene dye derivatives, chlorophyll derivatives, tetrapyrrole structures, porphyrins, chlorins, bacteriochlorins, phthalocyanines, texaphyrins, prodrugs, aminolevulinic acids, phenothiaziniums, squaraine, boron compounds, transition metal complexes, hypericin, riboflavin, curcumin, titanium dioxide, psoralens, tetracyclines, flavins, riboflavin, riboflavin derivatives, erythrosine, erythrosine derivatives, indocyanine green, and rose bengal.

9. The system of claim 1, wherein a concentration of each photosensitizer in the composition is from 0.01 μM to 1,000 μM.

10. The system of claim 1, wherein the light source includes an artificial light source or sunlight.

11. A composition, comprising:
at least two photosensitizers selected from the group consisting of methylene blue, riboflavin, erythrosine, rose bengal, and indocyanine green.

12. The composition of claim 11, wherein the composition is a solution including water, saline, or an alcohol.

13. The composition of claim 11, wherein a concentration of each photosensitizer in the composition is from 0.01 μM to 1,000 μM.

14. The composition of claim 11, wherein a concentration of each photosensitizer in the composition is from 0.1 μM to 1,000 μM.

15. The composition of claim 11, wherein a concentration of each photosensitizer in the composition is from 1 μM to 1,000 μM.

16. The composition of claim 11, wherein a concentration of each photosensitizer in the composition is from 10 μM to 1,000 μM.

17. The composition of claim 11, wherein a concentration of each photosensitizer in the composition is from 100 μM to 1,000 μM.

18. The composition of claim 11, comprising at least three photosensitizers selected from the group consisting of methylene blue, riboflavin, erythrosine, rose bengal, and indocyanine green.

19. A method for making a composition including two or more photosensitizers, comprising:
obtaining a baseline antimicrobial efficacy of a baseline composition including a single photosensitizer at a given concentration and given light parameters including illumination time, fluence rate, and lux;
making a combination composition including the single photosensitizer and one or more photosensitizers;
testing the combination composition for antimicrobial efficacy under one of the conditions:
a total concentration of photosensitizers of the combination composition is less than the given concentration of the baseline composition;
an illumination time is less than the illumination time of the baseline composition;
a fluence rate is less than the fluence rate of the baseline composition; and
a lux is less than the lux of the baseline composition.

20. The method of claim 19, further comprising, when the antimicrobial efficacy of the combination composition tests less than the baseline antimicrobial efficacy, replacing a photosensitizer other than the single photosensitizer with a different photosensitizer, and retesting the combination composition for antimicrobial efficacy under one of the following conditions:
a total concentration of photosensitizers of the combination composition is less than the given concentration of the baseline composition;
an illumination time is less than the illumination time of the baseline composition;
a fluence rate is less than the fluence rate of the baseline composition; and
a lux is less than the lux of the baseline composition

21. The method of claim 19, further comprising, when the antimicrobial efficacy of the combination composition tests greater than the baseline antimicrobial efficacy, making the combination composition into a disinfecting composition.

22. A disinfection system, comprising:
a light source that emits different wavebands of light at different fluence rates; and
an article incorporating a composition inside or on a surface of the article being exposed to the light source, wherein the composition includes a combination of at least two photosensitizers, wherein each of the at least two photosensitizers absorbs light of a different waveband emitted from the light source.

23. The disinfection system of claim 22, wherein the light source is a white light source.

24. The disinfection system of claim 22, wherein the light source is an LED emitting light in a blue waveband, yellow-green wavebands, and red waveband, wherein the red waveband has a lowest fluence rate compared to the blue waveband and the yellow-green wavebands.
